(19) United States
(12) Patent Application Publication
(10) Pub. No.: US 2018/0280364 A1
(43) Pub. Date: Oct. 4, 2018

(54) PHARMACEUTICAL COMPOSITIONS OF SODIUM PICOSULFATE, MAGNESIUM OXIDE AND CITRIC ACID

(71) Applicant: Hetero Labs Limited, Hyderabad (IN)

(72) Inventors: BANDI PARTHASARADHI REDDY, Hyerabad (IN); Podile Khadgapathi, Hyderabad (IN); Sunil Deviprasad Tiwari, Hyderabad (IN); Shetiya Prakash, Hyderabad (IN); Patchigolla Satyanarayana Rao, Hyderabad (IN); Chidara Ravi Chandra Gupto, Hyderabad (IN)

(21) Appl. No.: 15/933,166

(22) Filed: Mar. 22, 2018

(30) Foreign Application Priority Data
Mar. 22, 2017 (IN) 201741009969

(51) Int. Cl. A61K 31/4402; A61K 47/12; A61K 47/14; A61K 47/44; A61K 9/16

(52) U.S. Cl. CPC A61K 31/4402; A61K 47/12; A61K 47/14; A61K 31/194; A61K 9/1682; A61K 2300/00; A61K 47/44

(57) ABSTRACT

The present invention relates to stable pharmaceutical compositions of sodium picosulfate, magnesium oxide and citric acid. The invention also relates to processes for the preparation of such compositions and its use for bowel cleansing.

The following specification particularly describes the invention and the manner in which it is to be performed.

### FIELD OF THE INVENTION

The present invention relates to pharmaceutical compositions of sodium picosulfate, magnesium oxide and citric acid. The invention also relates to processes for the preparation of such compositions.

### BACKGROUND OF THE INVENTION

A pharmaceutical product used for cleansing of the colon as a preparation for colonoscopy, is presently sold under the trade name of Prepopik™ in the United States as powder for oral solution. Prepopik™ contains sodium picosulfate, magnesium oxide and anhydrous citric acid.

Compatibility study of sodium picosulfate with citric acid has been performed, which produced a wet mass with high known impurities and it demonstrates that sodium picosulfate is not compatible or stable with citric acid physically and chemically. As sodium picosulfate is having sensitivity towards an acidic nature ingredient, there is a need to formulate the composition of sodium picosulfate not in direct contact with the citric acid.

U.S. Pat. No. 8,481,083 claims process of making powder for oral solution of sodium picosulfate comprising the steps of: (a) a core of citric acid, coated with a layer of magnesium oxide, using simple mixing; and (b) another core of potassium bicarbonate coated with a layer of sodium picosulfate, using spray-coating technique.

The spray coating process is cumbersome, complicated and difficult to optimize. Hence, there is need to develop simplified process to prepare pharmaceutical compositions comprising sodium picosulfate, magnesium oxide and citric acid.

Accordingly, inventors of the present invention have developed the compositions using simple, cost effective and economical manufacturing process with improved stability.

### SUMMARY OF THE INVENTION

The present invention relates to pharmaceutical compositions of sodium picosulfate, magnesium oxide and citric acid.

One embodiment of the present invention relates to a pharmaceutical composition comprising sodium picosulfate and hydrophobic excipient.

Another embodiment of the present invention relates to a stable pharmaceutical composition in the form of powder or granules comprising: (i) blend of sodium picosulfate and a hydrophobic excipient; (ii) magnesium oxide and citric acid; and (iii) at least one pharmaceutically acceptable excipient.

Another embodiment of the present invention relates to a pharmaceutical composition in the form of powder or granules comprising: (i) blend of sodium picosulfate and a hydrophobic excipient selected from glyceryl dibehenate, glyceryl distearate, carnauba wax, hydrogenated vegetable oil and stearic acid; (ii) magnesium oxide and citric acid; and (iii) at least one pharmaceutically acceptable excipient selected from a basic agent, a sweetener, a flavor or a combination thereof.

Another embodiment of the present invention relates to process for preparing a pharmaceutical composition comprising the steps of: (i) blending sodium picosulfate with hydrophobic excipient and heating at elevated temperature for about 10 minutes, (ii) adding citric acid and magnesium oxide to the blend of step (i), and (iii) further adding at least one pharmaceutically acceptable excipient to the blend of step (ii) to get the powder or granular composition.

According to the present invention, upon heating the blend of sodium picosulfate and hydrophobic excipient together at elevated temperature, the hydrophobic excipient melts and forms a coat on the sodium picosulfate, which provides enhanced protection from degradation.

In another embodiment, the present invention relates to use of composition of the present invention for cleansing of the colon as a preparation for colonoscopy in adults.

In yet another embodiment, the present invention can also be used for other list of actives which are susceptible to degradation or incompatible with one or more other active or inactive ingredients.

### DETAILED DESCRIPTION OF THE INVENTION

The term “composition” as used herein refers to a dosage form suitable for oral administration, such as powder, granules, spheroids, pellets, pills, capsule, solution, suspension, emulsion and the like.

The term “pharmaceutically acceptable” as used herein means that which is useful in preparing a pharmaceutical composition that is generally safe and non-toxic.

The term “excipients” as used herein means a component of a pharmaceutical product that is not an active ingredient such as, for example, basic agents, sweeteners, flavors, diluents, carriers and the like. The excipients that are useful in preparing a pharmaceutical composition are generally safe and non-toxic.

As used in the specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The present invention relates to pharmaceutical compositions of sodium picosulfate, magnesium oxide and citric acid.

One aspect of the present invention relates to a pharmaceutical composition comprising sodium picosulfate and hydrophobic excipient.

Another aspect of the present invention relates to a stable pharmaceutical composition in the form of powder or granules comprising: (i) blend of sodium picosulfate and a hydrophobic excipient; (ii) magnesium oxide and citric acid; and (iii) at least one pharmaceutically acceptable excipient.

Another aspect of the present invention relates to a pharmaceutical composition in the form of powder or granules comprising: (i) blend of sodium picosulfate and a hydrophobic excipient selected from glyceryl dibehenate, glyceryl distearate, carnauba wax, hydrogenated vegetable oil and stearic acid; (ii) magnesium oxide and citric acid; and (iii) at least one pharmaceutically acceptable excipient selected from a basic agent, a sweetener, a flavor or a combination thereof.

Another embodiment of the present invention relates to process for preparing a pharmaceutical composition comprising the steps of: (i) blending sodium picosulfate with hydrophobic excipient and heating at elevated temperature for about 10 minutes, (ii) adding citric acid and magnesium oxide to the blend of step (i), and (iii) further adding at least one pharmaceutically acceptable excipient to the blend of step (ii) to get the powder or granular composition.

According to the present invention, upon heating the blend of sodium picosulfate and hydrophobic excipient together at elevated temperature, the hydrophobic excipient melts and forms a coat on the sodium picosulfate, which provides enhanced protection from degradation. The elevated temperature as used herein refers to the temperature at about 40° C. to about 100° C.

Generally, the hydrophobic excipient has a property of liquefying at a temperature from about 40° C. to about 100° C., that is to say, the melting point of respective hydrophobic excipient. Once the hydrophobic excipient liquefies, it spreads uniformly over the substrate as liquefied material will have better spreading property.

Hydrophobic excipient of the present invention includes water insoluble non-polymeric excipient selected from glyceryl behenate, glyceryl distearate, carnauba wax, stearic acid, hydrogenated vegetable oil, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, decenoic acid, docosanoic acid, spermaceti wax, Japan wax, bayberry wax, flax wax, beeswax, yellow wax, Chinese wax, shellac wax, lanolin wax. sugarcane wax, candelilla wax, castor wax, paraffin wax, microcrystalline wax, petrolatum wax, carbowax, mineral waxes, glyceryl monostearate, glyceryl tristearate; glyceryl dipalmitate, glyceryl tripalmitate, glyceryl monopalmitate, glyceryl palmitostearate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate, polyglyceryl diisostearate, lauroyl macrogolglycerides, oleoyl macrogolglycerides, stearoyl macrogolglycerides, palmitic acid, lauric acid, myristic acid, cetyl alcohol, stearyl alcohol, and the like or combinations thereof.

Pharmaceutically acceptable excipients for use in the pharmaceutical composition of the present invention may include one or more basic agents, sweeteners, flavors, colorants, diluents, binders and disintegrants.

As used herein, the term “basic agent” includes but is not limited to one or more of potassium bicarbonate, sodium bicarbonate, calcium carbonate, sodium carbonate and lithium carbonate and the like or combinations thereof.

Suitable sweeteners include, but are not limited to saccharides such as aspartame, sugar derivatives, sodium saccharin, sugarless sweeteners, hydrogenated starch hydrolysates, alone or in combination.

Suitable flavors include, but are not limited to orange, apple, pear, peach, vanilla, strawberry, cherry, apricot, watermelon, banana, cinnamon, wintergreen, eucalyptus, spearmint, peppermint, menthol and the like or combinations thereof.

Another aspect of the present invention relates to process for preparing pharmaceutical composition of sodium picosulfate, magnesium oxide and citric acid comprising:

(i) blending the sodium picosulfate with hydrophobic excipient and loading into suitable equipment and then heating at elevated temperature for about 10 minutes,

(ii) sieving a part of magnesium oxide, basic agent, sweetener and flavor separately, followed by blending for 10 minutes,

(iii) sieving anhydrous citric acid and remaining part of magnesium oxide separately, followed by blending for 30 minutes,

(iv) blending the material of step (i), (ii) and (iii) and finally, filling, in to packets.

According to the present invention, suitable equipments for healing include fluid bed dryer, tray dryer, spray dryer, fluid bed processor, fluid bed coaler, rapid mixer granulator, hot melt granulator, hot melt extruder, single pot granulator or processor, etc.

In another aspect, the present invention relates to use of composition of the present invention for cleansing of the colon as a preparation for colonoscopy in adults.

In yet another aspect, the present invention can also be used for other list of actives which are susceptible to degradation or incompatible (physically or chemically) with one or more other active or inactive ingredients.

### EXAMPLES

Certain specific aspects and embodiments of this invention are described in further detail by the examples below, which are provided only for purposes of illustration and are not intended to limit the scope of the invention in any manner.

### Comparative Example 1

### Composition of Sodium Picosulfate, Magnesium Oxide and Citric Acid as per U.S. '083 Patent Process

| Ingredient | mg/unit |
|---|---|
| Sodium picosulfate | 10.00 |
| Magnesium oxide light | 3500.00 |
| Anhydrous citric acid | 12000.00 |
| Potassium bicarbonate | 500.00 |
| Sodium saccharin | 45.00 |
| Orange flavor | 45.00 |
| Total weight per packet | 16100.00 |

### Brief Manufacturing Procedure

(i) Potassium bicarbonate core coated with a layer of sodium picosulfate, using spray coating process, followed by drying to get sodium picosulfate coated granules,

(ii) citric acid core coated with magnesium oxide,

(iii) materials of step (i) and (ii) were blended and finally, filled in to packets.

TABLE 1
Stability study:

| | Specification | | 40° C./75% RH |
|---|---|---|---|
| Impurity | limit | Initial | 1 month |
| RC-A | 0.5% | ND* | 0.027 |
| Bis Phenol IMP | 0.5% | ND | 0.009 |
| Highest Unknown | 0.2% | 0.299 | 1.269 |
| Total | 2.5% | 0.593 | 1.472 |

*Not detected

Observations: Composition of Comparative Example 1 was evaluated for stability. As can be seen from Table 1, known impurities were within the specification limits, but the unknown impurities were at higher levels as compared to predefined specification limits for the product.

### Comparative Example 2

### Composition of Sodium Picosulfate, Magnesium Oxide and Citric Acid by Simple Mixing

| Ingredient | mg/unit |
|---|---|
| Sodium picosulfate | 10.00 |
| Magnesium oxide light | 3500.00 |
| Anhydrous citric acid | 12000.00 |
| Potassium bicarbonate | 500.00 |
| Sodium saccharin | 45.00 |
| Orange flavor | 45.00 |
| Total weight per packet | 16100.00 |

### Brief Manufacturing Procedure

Sodium picosulfate, magnesium oxide light, anhydrous citric acid, potassium bicarbonate, sodium saccharin and orange flavor were blended together and filled in to packets.

TABLE 2
Stability study:

| | Specification | | 40° C./75% RH |
|---|---|---|---|
| Impurity | limit | Initial | 1 month |
| RC-A | 0.5% | 0.138 | 5.308 |
| Bis Phenol IMP | 0.5% | ND* | 11.586 |
| Highest Unknown | 0.2% | 0.075 | 1.536 |
| Total | 2.5% | 0.213 | 18.603 |

*Not detected

Observations: Composition of Comparative Example 2 was evaluated for stability. As can be seen from Table 2, known and unknown impurities were at higher levels as compared to predefined specification limits for the product, which may be due to direct interaction of sodium picosulfate with anhydrous citric acid as they are chemically incompatible. As evident from above impurity results, the said process may not be suitable for the product.

### Example 1

### Composition of Sodium Picosulfate, Magnesium Oxide and Citric Acid with Glyceryl Dibehenate

| Ingredient | mg/unit |
|---|---|
| Sodium picosulfate | 10.00 |
| Glyceryl dibehenate | 2.00 |
| Magnesium oxide light | 3500.00 |
| Anhydrous citric acid | 12000.00 |
| Potassium bicarbonate | 500.00 |
| Sodium saccharin | 45.00 |
| Orange flavor | 45.00 |
| Total weight per packet | 16102.00 |

### Brief Manufacturing Procedure

(i) Sodium picosulfate was blended with glyceryl dibehenate and the blend was loaded info fluid bed dryer and heated at 90° C. for about 10 minutes,

(ii) a part of magnesium oxide light, potassium bicarbonate, sodium saccharin and orange flavor were blended,

(iii) citric acid and remaining part of magnesium oxide light were blended,

(iv) materials of step (i), (ii) and (iii) were blended and finally filled in to packets.

TABLE 3
Stability study:

| | Specification | | 60° C. | 40° C./75% RH, | |
|---|---|---|---|---|---|
| Impurity | limit | Initial | 1 month | 1 month | 2 months |
| RC-A | 0.5% | 0.33 | 0.058 | 0.039 | 0.030 |
| Bis Phenol IMP | 0.5% | ND* | 0.016 | ND* | ND* |
| Highest Unknown | 0.2% | 0.047 | 0.101 | 0.054 | 0.053 |
| Total | 2.5% | 0.080 | 0.175 | 0.093 | 0.083 |

*Not Detected

Observations: Composition of Example 1 prepared using simplified process by treating sodium picosulfate with glyceryl dibehenate and evaluated for stability. As can be seen from Table 3, known and unknown impurities were at lower levels and within the limits as compared to predefined specification limits for the product at initial stage and after 1 month and 2 months of stability study.

### Example 2

### Composition of Sodium Picosulfate, Magnesium Oxide and Citric Acid by Treating with Glyceryl Distearate

| Ingredient | mg/unit |
|---|---|
| Intragranular materials: | |
| Sodium picosulfate | 10.00 |
| Glyceryl distearate | 2.00 |
| Extragranular materials: | |
| Magnesium oxide light | 3500.00 |
| Anhydrous citric acid | 12000.00 |
| Potassium bicarbonate | 500.00 |
| Sodium saccharin | 45.00 |
| Orange flavor | 45.00 |
| Total weight per packet | 16102.00 |

### Brief Manufacturing procedure

(i) Sodium picosulfate was blended with glyceryl distearate and the blend was loaded into rapid mixer granulator and heated at 80° C. for about 10 minutes,

(ii) a part of magnesium oxide light, potassium bicarbonate, sodium saccharin and orange flavor were blended,

(iii) citric acid and remaining part of magnesium oxide light were blended,

(iv) materials of step (i), (ii) and (iii) were blended and finally filled in to packets.

### Example 3

### Composition of Sodium Picosulfate, Magnesium Oxide and Citric Acid by Treating with Carnauba Wax

| Ingredient | mg/unit |
|---|---|
| Intragranular materials: | |
| Sodium picosulfate | 10.00 |
| Carnauba wax | 1.50 |
| Extragranular material: | |
| Magnesium oxide light | 3500.00 |
| Anhydrous citric acid | 12000.00 |
| Potassium bicarbonate | 500.00 |
| Sodium saccharin | 45.00 |
| Orange flavor | 45.00 |
| Total weight per packet | 16101.50 |

### Brief Manufacturing Procedure

(i) Sodium picosulfate was blended with carnauba wax and the blend was heated at required elevated temperature for about 10 minutes in suitable equipment.

(ii) a part of magnesium oxide light, potassium bicarbonate, sodium saccharin and orange flavor were blended,

(iii) citric acid and remaining part of magnesium oxide light were blended,

(iv) materials of step (i), (ii) and (iii) were blended and finally filled in to packets.

### Example 4

### Composition of Sodium Picosulfate, Magnesium Oxide and Citric Acid by Treating with Hydrogenated Vegetable Oil

| Ingredient | mg/unit |
|---|---|
| Intragranular materials: | |
| Sodium picosulfate | 10.00 |
| Hydrogenated vegetable oil | 3.00 |
| Extragranular materials: | |
| Magnesium oxide light | 3500.00 |
| Anhydrous citric acid | 12000.00 |
| Potassium bicarbonate | 500.00 |
| Sodium saccharin | 45.00 |
| Orange flavor | 45.00 |
| Total weight per packet | 16103.00 |

(i) Sodium picosulfate was blended with hydrogenated vegetable oil and the blend was heated at 80° C. for about 10 minutes in suitable equipment,

(ii) a part of magnesium oxide light, potassium bicarbonate, sodium saccharin and orange flavor were blended,

(iii) citric acid and remaining part of magnesium oxide light were blended,

(iv) materials of step (i), (ii) and (iii) were blended and finally filled in to packets.

### Example 5

### Composition of Sodium Picosulfate, Magnesium Oxide and Citric Acid by Treating with Stearic Acid

| Ingredient | mg/unit |
|---|---|
| Intragranular materials: | |
| Sodium picosulfate | 10.00 |
| Stearic acid | 2.00 |
| Extragranular materials: | |
| Magnesium oxide light | 3500.00 |
| Anhydrous citric acid | 1200.00 |
| Potassium bicarbonate | 500.00 |
| Sodium saccharin | 45.00 |
| Orange flavor | 45.00 |
| Total weight per packet | 16102.00 |

### Brief Manufacturing Procedure

(i) Sodium picosulfate was blended with stearic acid and the blend was heated at 75° C. for about 10 minutes,

(ii) a part of magnesium oxide light, potassium bicarbonate, sodium saccharin and orange flavor were blended,

(iii) citric acid and remaining part of magnesium oxide light were blended,

(iv) materials of step (i), (ii) and (iii) were blended and finally filled in to sachets.

1. A pharmaceutical composition comprising sodium picosulfate and hydrophobic excipient.

2. The pharmaceutical composition according to claim 1, wherein the hydrophobic excipient is selected from glyceryl dibehenate, glyceryl distearate, carnauba wax, hydrogenated vegetable oil and stearic acid.

3. The pharmaceutical composition according to claim 1, further comprises magnesium oxide and citric acid.

4. The pharmaceutical composition according to claim 1, is in the form of powder or granules.

5. A stable pharmaceutical composition in the form of powder or granules comprising:
(i) blend of sodium picosulfate and a hydrophobic excipient;
(ii) magnesium oxide and citric acid; and
(iii) at least one pharmaceutically acceptable excipient.

6. A process for preparing the pharmaceutical composition according to claim 5, comprising the steps of:
(i) blending sodium picosulfate with hydrophobic excipient,
(ii) heating blend of step (i) at elevated temperature for about 10 minutes,
(iii) adding citric acid and magnesium oxide to the blend of step (ii), and
(iv) further adding at least one pharmaceutically acceptable excipient to the blend of step (iii) to get the powder or granular composition.

7. The pharmaceutical composition according to claim 5, wherein the hydrophobic excipient is selected from glyceryl dibehenate, glyceryl distearate, carnauba wax, hydrogenated vegetable oil and stearic acid.

8. The pharmaceutical composition according to claim 1, further comprising at least one pharmaceutically acceptable excipient selected from the group consisting of a basic agent, a sweetener, a flavor and a combination thereof.

9. The pharmaceutical composition according to claim 5, wherein at least one pharmaceutically acceptable excipient selected from a basic agent, a sweetener, a flavor or a combination thereof.

10. A pharmaceutical composition in the form of powder or granules, comprising:
(i) a blend of sodium picosulfate and a hydrophobic excipient, wherein the hydrophobic excipient is selected from the group consisting of glyceryl dibehenate, glyceryl distearate, carnauba wax, hydrogenated vegetable oil and stearic acid;
(ii) magnesium oxide and citric acid; and
(iii) at least one pharmaceutically acceptable excipient selected from a basic agent, a sweetener, a flavor or a combination thereof.
